(19) United States
(12) Patent Application Publication
(10) Pub. No.: US 2019/0223486 A1
(43) Pub. Date: Jul. 25, 2019

(54) CATECHIN ABSORPTION ENHANCER FOR ENHANCING CATECHIN ABSORPTION IN SMALL INTESTINAL EPITHELIAL CELLS

(71) Applicant: AMOREPACIFIC CORPORATION, Seoul (KR)

(72) Inventors: Jin Oh CHUNG, Yongin-si, Gyeonggi-do (KR); Su Kyung KIM, Yongin-si, Gyeonggi-do (KR); Jeong Kee KIM, Yongin-si, Gyeonggi-do (KR); Wan Gi KIM, Yongin-si, Gyeonggi-do (KR); Song Seok SHIN, Yongin-si, Gyeonggi-do (KR); Soon Mi SHIM, Seoul (KR); Seon Bong LEE, Seoul (KR); Ji Hoon SONG, Seoul (KR); Seung Beom SEO, Seoul (KR)

(73) Assignee: AMOREPACIFIC CORPORATION, Seoul (KR)

(21) Appl. No.: 16/336,797

(22) PCT Filed: Sep. 20, 2017
(86) PCT No.: PCT/KR2017/010295

§ 371 Date: Mar. 26, 2019

(30) Foreign Application Priority Data
Sep. 27, 2016 (KR) 10-2016-0124027

(51) Int. Cl. A23L 33/10; A61K 31/357; A61K 31/352

(52) U.S. Cl. CPC A23L 33/10; A23V 2200/32; A61K 31/352; A61K 31/357

(57) ABSTRACT

The present invention relates to a catechin absorption enhancer in small intestinal epithelial cells, and more particularly, to an enhancer for improving an absorption rate of catechin in small intestinal cells and a composition including the same. The composition including the catechin absorption enhancer in the small intestinal epithelial cells according to the invention can enhance the absorption rate of catechin in the small intestinal epithelial cells, which leads to improved bioavailability. Therefore, the present invention can be expected to improve an antioxidant effect, an anti-aging effect, a lipolytic effect, and a variety of other effects of the catechin.

### TECHNICAL FIELD

This application claims the benefits of Korean Patent Application No. 10-2016-0124027 filed on Sep. 27, 2016 with the Korean Intellectual Property Office (KIPO), the disclosure of which are incorporated by reference in its entirety.

The present invention relates to a catechin absorption enhancer in small intestinal epithelial cells, and more particularly, to an enhancer for improving an absorption rate of catechin in small intestinal cells and a composition comprising the same.

### BACKGROUND ART

A main component of green tea showing various bioactivities is catechin included in the leaves of green tea. In recent years, there is a growing interest in such catechin because it has been reported that it has various activities such as an antioxidant effect, an antiviral effect, an anticancer effect, an anti-allergic effect, and the like, particularly because it has been known that it has an excellent effect in reducing the body fat and weight. In general, catechin includes a total of eight types of catechins: (+)-catechin (C), (−)-epicatechin (EC), (−)-gallocatechin (GC), (−)-epigallocatechin (EGC), (−)-catechin gallate (CG), (−)-epicatechin gallate (ECG), (−)-gallocatechin gallate (GCG), and (−)-epigallocatechin gallate (EGCG).

However, the catechins have a poor rate of absorption into small intestinal epithelial cells and show the rapid metabolism and efflux in the small intestinal epithelial cells. Therefore, it is in effect known that the catechins are not highly effective due to its low bioavailability.

### PRIOR-ART DOCUMENTS

Patent Document 1: Korean Patent No. 2013-0036631 entitled “Agent for improvement of cathechin bioavailability”
Patent Document 2: Korean Patent No. 2014-0072307 entitled “Agent for improvement of catechin bioavailability comprising cyclodextrin”

### DISCLOSURE

### Technical Problem

An object of the present invention is to provide a catechin absorption enhancer for enhancing an absorption rate of catechin in small intestinal epithelial cells so as to improve bioavailability.

Another object of the present invention is to provide a composition comprising the catechin absorption enhancer.

### Technical Solution

In order to achieve the above object, the present invention provides a catechin absorption enhancer in small intestinal epithelial cells, which includes fisetin, quercetin, or a mixture thereof.

In addition, the present invention provides a pharmaceutical composition comprising catechin and the catechin absorption enhancer.

In addition, the present invention provides a health food composition comprising catechin and the catechin absorption enhancer.

### Advantageous Effects

A catechin absorption enhancer in small intestinal epithelial cells according to the present invention and a composition comprising the catechin absorption enhancer can enhance an absorption rate catechin in the small intestinal epithelial cells, which leads to improved bioavailability. Therefore, the present invention can be expected to improve an antioxidant effect, an anti-aging effect, a lipolytic effect, and a variety of other effects of the catechin.

### DESCRIPTION OF DRAWINGS

FIG. 1 is a conceptual diagram of a small intestine model according to Experimental Example 1 of the present invention.

FIG. 2 shows the data for the relative comparison of effects of a catechin composition including fisetin according to the present invention on an enhancement of a catechin absorption rate in a small intestine model.

FIG. 3 shows the data for the relative comparison of effects of a catechin composition including quercetin according to the present invention on an enhancement of a catechin absorption rate in the small intestine model.

FIG. 4 shows the data for the relative comparison of effects of a catechin composition including fisetin and quercetin according to the present invention on an enhancement of a catechin absorption rate in the small intestine model.

### BEST MODE

In general, it is known that less than 2% of a catechin intake is absorbed by the intestines, and such catechins are considered to have low bioavailability because the catechins are sensitive to in vivo digestive fluid environments, have a low rate of absorption into small intestinal cells, and show the rapid metabolism and efflux in the small intestine. This is based on facts that the catechins are mainly degraded in an upper portion of the small intestine whose pH is high in the course of digestion and in which active oxygen is present, and that the catechins are reversely transported into the intestines without penetrating through the small intestinal epithelial cells by means of reverse transport proteins such as P-glycoprotein.

In recent years, it has been known that, when glycocomponents such as sugar are added to a green tea extract including catechins, the catechins show an increased stability in the digestive organs and have a high catechin accumulation rate in small intestinal epithelial cells, indicating that the addition of the glycocomponents causes an increase in in vivo absorption rate of the catechins. However, the glycocomponents such as sugar have a problem in that they are low-nutrient and high-calorie components, and thus have a negative effect on the health of some users. Therefore, there is a need for alternative methods capable of enhancing the absorption rate of catechins in the small intestinal epithelial cells.

Therefore, the present invention provides a catechin absorption enhancer in small intestinal epithelial cells, which includes fisetin, quercetin, or a mixture thereof.

Catechin is a kind of polyphenols used as a meaning of generally referring to catechins, and types of such catechins include (+)-catechin (C), (−)-epicatechin (EC), (−)-gallocatechin (GC), (−)-epigallocatechin (EGC), (−)-catechin gallate (CG), (−)-epicatechin gallate (ECG), (−)-gallocatechin gallate (GCG), and (−)-epigallocatechin gallate (EGCG). In the present invention, “catechin” is used as a meaning of referring to part or all of the catechins listed above.

In the present invention, catechin may be included in the form of a green tea extract in a composition. Preferably, the green tea extract may include the catechin at 20% by weight or more, more preferably at least 30 to 50% by weight, based on the total weight of the extract.

In the present invention, fisetin included as the catechin absorption enhancer in the small intestinal epithelial cells is a substance represented by the following Formula 1, and is a compound whose CAS No. is 528-48-3, that is, 3,7,3′,4′-tetrahydroxyflavone.

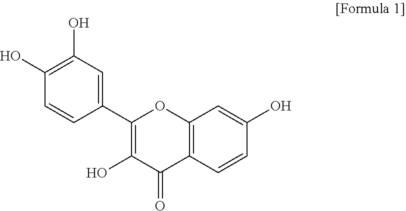 [SMILES: O=c1c(O)c(-c2ccc(O)c(O)c2)oc2cc(O)ccc12]

Fisetin is a component found in strawberry, apple, grape, onion, mango, and acacia as a unique polyphenolic flavonoid, and has been studied for potent anti-inflammatory, anticancer, antiviral and antioxidant effects. In particular, it was reported that fisetin has high neuroprotective and memory-improving effects.

In the present invention, quercetin included as the catechin absorption enhancer in the small intestinal epithelial cells is a substance represented by the following Formula 2, and is a compound whose CAS No. is 528-48-3, that is, 3,3′,4′,5,7-pentahydroxyflavone.

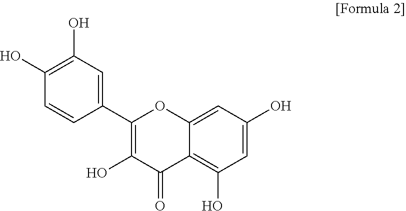 [SMILES: O=c1c(O)c(-c2ccc(O)c(O)c2)oc2cc(O)cc(O)c12]

Quercetin is one of flavonoids known to be abundantly present in red wine, onion, kiwi fruit, green tea, apple, berries, the heart of cabbage (cabbage, broccoli, cauliflower, turnip, etc.). Quercetin is known to have various bioactivities to remove heavy metals, reduce blood cholesterols, and prevent adult diseases such as hypertension and diabetes and also have antioxidative actions and antiviral activities. Also, quercetin is an antioxidant known to protect tissues from materials oxidizing cells in the body, be effect for anti-inflammatory and anticancer actions, thereby showing an anticancer effect on breast cancer, colon cancer, ovarian cancer, gastric cancer, bladder cancer, etc.

In the present invention, when necessary, catechin, fisetin or quercetin may be used in the form of pharmaceutically and sitologically acceptable salts or hydrates or prodrugs thereof. Also, the aforementioned compounds are mainly extracted from plants, and thus are low toxic and not pungent to human bodies.

The catechin absorption enhancer in small intestinal epithelial cells according to the present invention may include the fisetin and the quercetin alone or may include the fisetin and the quercetin in the form of a mixture thereof. When the fisetin and the quercetin are included as the mixture of fisetin and quercetin, an absorption rate of catechin in small intestinal epithelial cells is further improved, compared to when the fisetin and the quercetin are included alone. In this case, a weight ratio of the mixture fisetin and the quercetin may be in a range of 0.1:9.9 to 9.9:0.1, preferably in a range of 1:2 to 2:1, and more preferably in a range of 1:1.

Catechin was reported in the prior art to have various effects such as an antioxidant effect, an antiviral effect, an anticancer effect, an anti-allergic effect, etc., but did not secure satisfactory effects and efficacy due to low bioavailability in the small intestinal cells. Accordingly, when the catechin absorption enhancer including the fisetin and/or quercetin provided herein is applied, the catechin absorption enhancer may enhance the bioavailability of catechins in the small intestine, thereby making it possible to secure the effects and efficacy.

The present invention provides a pharmaceutical composition, characterized by including catechin; and one catechin absorption enhancer selected from fisetin, quercetin, or a combination thereof.

Also, the present invention provides a health food composition, characterized by including catechin; and one catechin absorption enhancer selected from fisetin, quercetin, or a mixture thereof.

According to the present invention, the catechin absorption enhancer is included at 0.1 to 50% by weight, based on the total weight of the catechin. More preferably, when the catechin absorption enhancer includes the fisetin or quercetin alone, the catechin absorption enhancer is included at 5 to 20% by weight, based on the total weight of the catechin. When the catechin absorption enhancer includes the fisetin and quercetin as a mixture thereof, the catechin absorption enhancer is included at 1 to 10% by weight, based on the total weight of the catechin. When the content of the catechin absorption enhancer falls within this content range, the catechin absorption enhancer is suitable for exhibiting an desired effect of the present invention and also may satisfy both stability and safety of the composition, and thus desirable in aspects of cost effectiveness.

In addition to the aforementioned components, the pharmaceutical composition or health food composition according to the present invention may further include another component capable of enhancing the bioavailability of catechins at a level of quantity that does not hinder the efficacy of the catechin. For example, the pharmaceutical composition or health food composition may include any one of sugars, acids, and sugar alcohols.

The pharmaceutical composition or health food composition may be effective in losing the body weight, reducing body fats, and preventing and ameliorating obesity as well. Also, the pharmaceutical composition may have an excellent antioxidant activity and reverse an aging process.

The pharmaceutical composition according to the present invention may be orally administered in the form of a solid, semisolid or liquid phase after a commercially available inorganic or organic carrier is added thereto. A preparation for oral administration may include tablets, pills, soft and hard capsules, powders, grains, granules, solutions, emulsions, syrups, pellets, and the like.

The pharmaceutical composition may be readily formulated according to methods generally known in the art. In this case, a surfactant, an excipient, a pigmenting agent, a seasoning agent, a preservative, a stabilizing agent, a buffering agent, a suspending agent, or other commercially available adjuvants may be used in suitable amounts.

Also, the doses of the active components may vary depending on the age, sex, and weight of a subject to be treated, and a disease and pathological conditions to be treated, a route of administration, or the prescriber's judgement. The dosage determined based on these factors is within a range determined by those skilled in the art. For example, catechin may be administered once or three times a day at 100 to 1,000 mg, preferably 300 to 500 mg. In this case, the dose of the catechin is not intended to limit the scope of the present invention by means of any methods.

The pharmaceutical composition may be administered as an individual therapeutic agent or administered in combination with other therapeutic agents, and may be administered sequentially or concomitantly with conventional therapeutic agents. It is important to administer the pharmaceutical composition at an amount that can exhibit the maximum effect when used in a minimal amount without causing any side effects in consideration of all the aforementioned factors. In this case, the amount of the pharmaceutical composition may be readily determined by those skilled in the art.

The health food composition according to the present invention may be a heath food composition, a functional food composition, and a food additive composition. The compositions may be applied to various formulations such as tablets, pills, capsules, granules, drinks, caramels, diet bars, tea bags, and the like using conventional method which includes adding various types of excipients or additives. In addition to the active components, a person having ordinary skill in the art may properly select and blend components generally used in the related art without any difficulty, depending on the formulations and purposes of use. In this case, such components may have a synergy effect when bended with other components.

### MODE FOR INVENTION

Hereinafter, the present invention will be described in further detail with reference to examples and experimental examples thereof for better understanding of the present invention. However, it should be understood that the example and experimental examples of the present invention may be modified into various other forms, and are not intended to limit the scope of the present invention to example and experimental examples described below. The examples of the present invention will be provided to more completely explain the present invention to persons of ordinary skill in the art.

### Example 1

A composition was prepared by mixing catechin and fisetin at such a mixing ratio that the fisetin was present at 5% by weight, 10% by weight, and 20% by weight, based on the total weight of the catechin. In this case, the catechin includes four types of epicatechin (EC), epigallocatechin (EGC), epicatechin gallate (ECG), and epigallocatechin gallate (EGCG), and their content ratios are identical to the ratios of four catechins in a conventional green tea extract. That is, these catechins were included at 200 ppm, 600 ppm, 150 ppm, and 850 ppm, respectively.

### Example 2

A composition was prepared using quercetin instead of the fisetin used in Example 1.

### Example 3

A composition was prepared using a mixture of fisetin and quercetin instead of the fisetin used in Example 1. In this case, a content ratio of the fisetin and quercetin was set to be in a range of 1:1.

### Experimental Example 1

To an in vivo absorption rate, Caco-2 cells were generally used as human small intestinal cells. As media used for cell culture and sample treatment, Dullbeco's modified eagle media (DMEM, Corning) supplemented with 10% fetal bovine serum (FBS, Biotechnics research), 1% non-essential amino acids (Sigma), 1% penicillin/streptomycin (Corning), and 0.1% gentamycin (Sigma) were used.

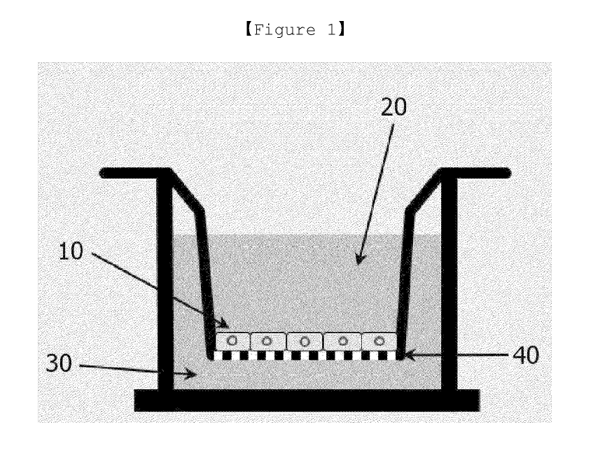

To apply an in vitro model similar to a human small intestine so as to check an absorption rate of catechin in small intestinal cells, a 12-well transwell (Collagen coated, Corning) was used in this experiment. More particularly, as shown in FIG. 1, a small intestine model was established, and Caco-2 cells 10 were injected into an apical part 20, and then cultured at 37° C. for 2 to 3 weeks under a 5% CO₂ atmosphere. In this case, the media were replaced every 2 days. After 2 to 3 weeks of the injection of the Caco-2 cells into the apical part 20, cells in which a value measured as a TEER resistance value was greater than 250Ω were selected and used for this experiment. Thereafter, an apical part 20, in which the Caco-2 cells underlay on a membrane of polytetrafluoroethylene (PTFE) 40 as a single layer in each treated group, was treated with each of the sample compositions prepared in Examples 1 to 3, and then cultured for 2 hours under a 5% CO₂ atmosphere. Then, a medium of a basolateral part 30 was taken and diluted at 1:1 (v:v) with methanol (MeOH), sonicated for 10 seconds, filtered through a 0.45 μm syringe, and then immediately analyzed using UPLC-PDA-ESI-MS/MSn.

Results

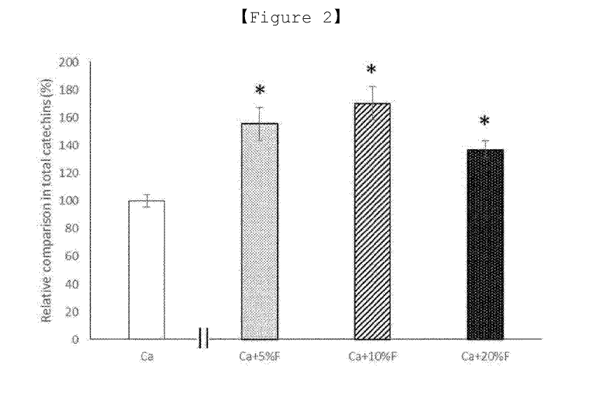

FIG. 2 shows the data for the relative comparison of effects of a catechin composition including fisetin (Example 1) on an enhancement of a catechin absorption rate in a small intestine model. When the absorption rate of untreated catechin was assumed to be 100%, it was revealed that the absorption rate of the catechin composition was 160%, the value of which increased approximately 1.6-fold, when the catechin composition included 5% by weight of fisetin, 170%, the value of which increased approximately 1.7-fold, when the catechin composition included 10% by weight of fisetin, and 140%, the value of which increased approximately 1.4-fold, when the catechin composition included 20% by weight of fisetin. That is, it can be seen that the catechin composition containing 10% by weight of fisetin had the highest absorption rate.

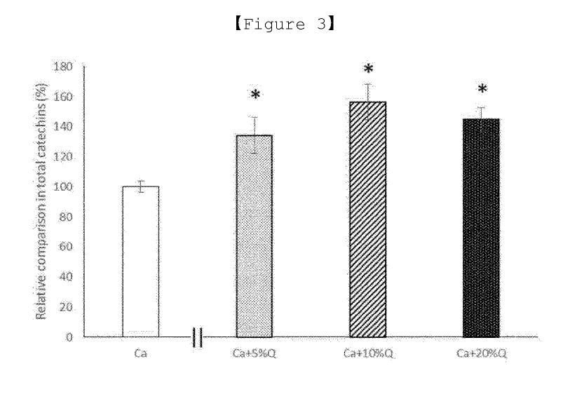

FIG. 3 shows the data for the relative comparison of effects of a catechin composition including quercetin (Example 2) on an enhancement of a catechin absorption rate in the small intestine model. When the absorption rate of the untreated catechin was assumed to be 100%, it was revealed that the absorption rate of the catechin composition was 135%, the value of which increased approximately 1.35-fold, when the catechin composition included 5% by weight of quercetin, 160%, the value of which increased approximately 1.6-fold, when the catechin composition included 10% by weight of quercetin, and 145%, the value of which increased approximately 1.45-fold, when the catechin composition included 20% by weight of quercetin. That is, it can be seen that the catechin composition containing 10% by weight of quercetin had the highest absorption rate.

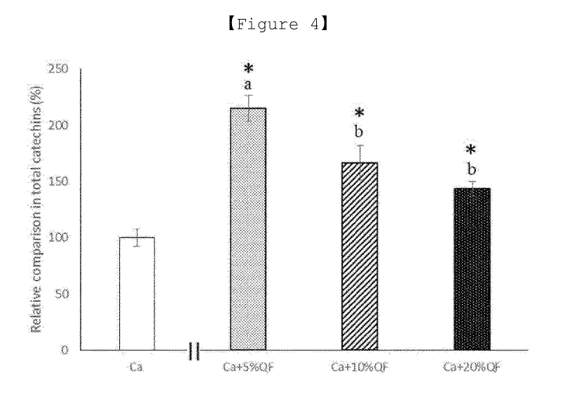

FIG. 4 shows the data for the relative comparison of effects of a catechin composition including fisetin and quercetin at a weight ratio of 1:1 (Example 3) on an enhancement of a catechin absorption rate in the small intestine model. When the absorption rate of the untreated catechin was assumed to be 100%, it was revealed that the absorption rate of the catechin composition was 220%, the value of which increased approximately 2.2-fold, when the catechin composition included 5% by weight of the fisetin/quercetin mixture, 165%, the value of which increased approximately 1.65-fold, when the catechin composition included 10% by weight of the fisetin/quercetin mixture, and 145%, the value of which increased approximately 1.45-fold, when the catechin composition included 20% by weight of the fisetin/quercetin mixture.

Based on the experimental results, it can be seen that the composition containing each of fisetin and quercetin at a content of 10% by weight had the highest catechin absorption rate in the case of Examples 1 and 2 in which the fisetin and the quercetin were added alone. Also, it can be seen that the composition containing the mixture of fisetin and quercetin at a content of 5% by weight had the highest catechin absorption rate in the case of Example 3 in which the fisetin and the quercetin were added in the form of a mixture thereof.

Also, it was confirmed that, when comparing only the data for the compositions in which the fisetin, the quercetin, and the mixture of fisetin and quercetin were contained at 5% by weight, respectively, based on the total content of the catechin, the compositions containing fisetin and quercetin alone increased approximately 1.6-fold and approximately 1.35-fold, respectively, whereas the composition to which the mixture of fisetin and quercetin was added increased approximately 2.2-fold. Further, it can be seen that, when the fisetin and the quercetin were added at 5% by weight in the form of a mixture thereof, an increase (2.2-fold) in the highest catechin absorption rate was higher than increases (1.7-fold, 1.6-fold) in the highest catechin absorption rate when each of the fisetin and quercetin was added at 10% by weight. In conclusion, it can be seen that the catechin absorption enhancer included as the mixture of fisetin and quercetin had a synergy effect, compared to the catechin absorption enhancers including fisetin and quercetin alone.

Formulation examples of the composition according to one aspect of the present invention will be described below. However, it should be understood that the composition may be applied to various other formulations, the contents of which are intended to describe the present invention in detail, but not intended to limit the scope of the present invention.

### [Formulation Example 1] Healthy Drinks

10% by weight of catechin, 1% by weight of a catechin absorption enhancer, 10% by weight of enzymatically modified stevia, 10% by weight of a grapefruit concentrate, and the balance of purified water were mixed, and the resulting mixture was heated at 80 to 90° C. for an hour while stirring according to a conventional method of prepare a healthy drink. Thereafter, the resulting solution was filtered, and packed into a sterile vessel, which was hermetically sealed, sterilized, and kept refrigerated. This solution was then used for preparation of healthy drink compositions.

### [Formulation Example 2] Pills

28% by weight of catechin, 2% by weight of a catechin absorption enhancer, 10% by weight of citric acid, 10% by weight of xylitol, 10% by weight of corn starch, 20% by weight of glycerin, and 20% by weight of sorbitol were mixed, and the resulting mixture was prepared into pills using a pill-making machine.

### [Formulation Example 3] Tablets

37% by weight of catechin, 3% by weight of a catechin absorption enhancer, 10% by weight of ascorbic acid, 7.5% by weight of xylitol, 20% by weight of dextrin, and 20% by weight of crystalline cellulose were mixed, and then granulated using a fluidized bed dryer. Thereafter, 2.5% by weight of sugar ester was added thereto, and the resulting mixture was compressed into tablets using a tableting machine.

### [Formulation Example 4] Granules

18% by weight of catechin, 2% by weight of a catechin absorption enhancer, 10% by weight of ascorbic acid, 10% by weight of xylitol, 5% by weight of enzymatically modified stevia, and 55% by weight of isomalt were mixed, and then shaped into granules using a fluidized bed dryer. Thereafter, the granules were packed into bags.

### BRIEF DESCRIPTION OF PARTS IN THE DRAWINGS

10. Caco-2 cells
20. Apical part
30. Basolateral part
40. PTFE membrane

1. A catechin absorption enhancer in small intestinal epithelial cells, comprising fisetin, quercetin, or a mixture thereof.

2. The catechin absorption enhancer of claim 1, wherein a weight ratio of the mixture fisetin and the quercetin is in a range of 0.1:9.9 to 9.9:0.1.

3. A pharmaceutical composition comprising:
catechin; and
one catechin absorption enhancer selected from the group consisting of fisetin, quercetin, and a mixture thereof.

4. The pharmaceutical composition of claim 3, wherein the catechin comprises at least one selected from the group consisting of (+)-catechin (C), (−)-epicatechin (EC), (−)-gallocatechin (GC), (−)-epigallocatechin (EGC), (−)-catechin gallate (CG), (−)-epicatechin gallate (ECG), (−)-gallocatechin gallate (GCG), and (−)-epigallocatechin gallate (EGCG).

5. The pharmaceutical composition of claim 3, wherein the catechin absorption enhancer is included at 0.1 to 50% by weight, based on the total weight of the catechin.

6. The pharmaceutical composition of claim 3, wherein the catechin absorption enhancer comprises 5 to 20% by weight of the fisetin or the quercetin, based on the total weight of the catechin.

7. The pharmaceutical composition of claim 3, wherein the catechin absorption enhancer comprises 1 to 10% by weight of the mixture of fisetin and quercetin, based on the total weight of the catechin.

8. A health food composition comprising:
catechin; and
one catechin absorption enhancer selected from the group consisting of fisetin, quercetin, and a mixture thereof.

9. The health food composition of claim 8, wherein the catechin comprises at least one selected from the group consisting of (+)-catechin (C), (−)-epicatechin (EC), (−)-gallocatechin (GC), (−)-epigallocatechin (EGC), (−)-catechin gallate (CG), (−)-epicatechin gallate (ECG), (−)-gallocatechin gallate (GCG), and (−)-epigallocatechin gallate (EGCG).

10. The health food composition of claim 8, wherein the catechin absorption enhancer is included at 0.1 to 50% by weight, based on the total weight of the catechin.

11. The health food composition of claim 8, wherein the catechin absorption enhancer comprises 5 to 20% by weight of the fisetin or the quercetin, based on the total weight of the catechin.

12. The health food composition of claim 8, wherein the catechin absorption enhancer comprises 1 to 10% by weight of the mixture of fisetin and quercetin, based on the total weight of the catechin.
